(19) United States
(12) Patent Application Publication
(10) Pub. No.: US 2026/0041622 A1
(43) Pub. Date: Feb. 12, 2026

(54) Reduction of Signs of Skin Aging

(71) Applicant: CarboCode S.A., Cantanhede (PT)

(72) Inventors: Cornelia PYKO, Cantanhede (PT); Feryel ADAM, Cantanhede (PT); Bruce MCCONNELL, Cantanhede (PT)

(21) Appl. No.: 19/100,872

(22) PCT Filed: Aug. 4, 2023
(86) PCT No.: PCT/IB2023/057918

§ 371 Date: Feb. 3, 2025

(30) Foreign Application Priority Data
Aug. 5, 2022 (PT) 118148
Oct. 10, 2022 (PT) 118249
Nov. 15, 2022 (PT) 118344

(51) Int. Cl. A61K 8/68; A61Q 19/00; A61Q 19/08

(52) U.S. Cl. CPC A61K 8/68; A61Q 19/004; A61Q 19/007; A61Q 19/08; A61K 2800/5922; A61K 2800/92

(57) ABSTRACT

Cosmetic use of a composition for reducing and/or ameliorating signs of skin aging in an individual, wherein the composition comprises a compound of formula I, wherein the carbon-carbon bond noted Formula II is a double or a single bond, R¹ is an alkyl chain having 10-20 carbon atoms, R² is hydrogen or a substituted or unsubstituted acyl having 16-34 carbon atoms, R³ is hydrogen when the carbon-carbon bond noted Formula II is a double or a single bond, or R³ is —OH when the carbon-carbon bond noted Formula II is a single bond, R⁴ is hydrogen when the carbon-carbon bond noted Formula II is a double or a single bond, or R⁴ is hydrogen or —OH when the carbon-carbon bond noted Formula II is a double bond, X is hydrogen or a glycosyl moiety, wherein the glycosyl moiety is a lactosyl moiety or a glucosyl moiety.

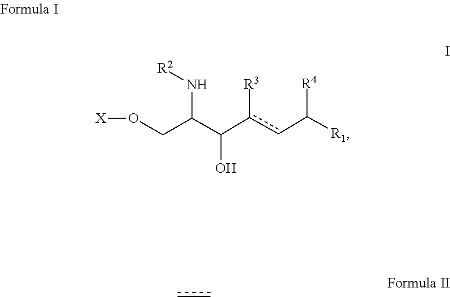

### FIELD OF THE INVENTION

The present invention relates to a use of a composition for reducing and/or ameliorating signs of skin aging in an individual.

### BACKGROUND OF THE INVENTION

The skin is the outer sheath of the body, which interfaces with the external environment. It is a complex structure made of several tissue layers, each with a distinct cellular composition. The upper layer of the skin is the epidermis, a stratified structure interspersed with hair follicles. The outer epidermal surface is made of cornified keratinocytes or corneocytes that form a dense cytoskeletal network of keratin filaments. The basal layer harbours the epidermal stem and progenitor cells. The dermis lies underneath the epidermis and consists of dermal fibroblasts that produce collagen, elastin and glycosaminoglycans that form the extracellular matrix (ECM), as well as the melanocytes that produce the photo-protective pigment melanin.

The dermal-epidermal junction (DEJ) forms the interface between the lower part of the epidermis and the top layer of the dermis. In addition to connecting dermis and epidermis, it protects against mechanical sheer, controls cellular organization and differentiation and provides a semipermeable barrier. The DEJ is made up of three layers, namely the hemidesmosome anchoring filament (including lamina lucida), the basement membrane (i.e. lamina densa) and anchoring fibril-layers. The DEJ is composed mainly of basal keratinocyte products, with a minor contribution from dermal fibroblasts. Within the hemidesmosomes are several macromolecules that attach the plasma membrane of the basal keratinocytes to the basement membrane. The basement membrane is composed primarily of laminins, type IV collagen and heparan sulfate proteoglycans.

Intercellular adhesion is formed by Cadherin-based adherens junctions (AJs) and desmosomes which are coupling the actin or intermediate filament cytoskeletons. As such, these intercellular junctions are essential to provide integrity to epithelia and other tissues. The primary function of desmosomes is strong adhesion, providing intercellular links in the desmosome-IF (intermediate filament) complex and lending great tensile strength to tissues that are subject to mechanical stress, such as the epidermis.

Skin aging is a complex biological process, leading to cumulative structural and physiologic alterations and progressive changes in each skin layer, as well as changes in skin appearance. Characteristics of skin aging include loss of hydration due to higher transepidermal water loss (TEWL), dryness, visible fine lines and wrinkles, uneven skin pigmentation, loss of elasticity and skin sagging. Both intrinsic and extrinsic factors affect the epidermal and dermal layers of the skin. Intrinsic aging processes are the result of chronological, inevitable senescence of the skin cells which varies depending on ethnicity, hormones and the anatomical region of the affected skin. Extrinsic skin aging is a result of all the external factors including lifestyle, smoking, UV exposure and the environment, which have a cumulative effect over time.

Thinning and flattening of the DEJ structure is commonly observed in aged skin, which results in a compromised adhesion of the epidermis onto the dermis and a consequent reduction of mechanical stability and structural integrity. The interface area through which nutrients and signaling molecules move is also reduced. These changes also applied to the intercellular junctions, which results in a weakened cell-cell adhesion but also cell-cell communication. Photoaged skin faces additional structural impairment, expressed as a disrupted and multilayered lamina densa. This impairment results from elevated expressions of extracellular matrix degrading enzymes, including matrix metalloproteinases (MMPs), urinary plasminogen-activator/plasmin and heparinase, which can also degrade the epidermal basement membrane comprising proteins. In addition to the structural impairment of the DEJ, age-related reductions of the basement membrane proteins comprising DEJ, including collagen IV, nidogen, integrin 34 and laminin-332, have been repeatedly reported.

With ever-rising life expectancies around the world, the physical appearance of aging is becoming an increasingly common cosmetic issue. Skin appearance plays a particularly crucial role for self-esteem and social interactions, and skin health and beauty are considered among the principal factors representing overall well-being and the perception of health in humans, Therefore, the demand for cosmetic products that may be applied to the skin to combat skin aging, especially facial skin aging, has grown enormously.

The recently identified importance of the DEJ on skin aging suggests a new anti-aging mode of action.

### SUMMARY OF THE INVENTION

The present inventors have surprisingly found certain compounds that efficiently reduce or ameliorate signs of skin aging. They are especially targeted at providing mechanical stability to the skin, particularly strengthening the DEJ structure and promoting cell adhesion and intercellular communication.

The present invention comprises the following aspects:
(1) Cosmetic use of a composition for reducing and/or ameliorating signs of skin aging in an individual, wherein the composition comprises a compound of formula I

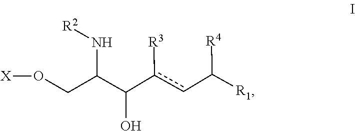

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.
(2) Method for reducing and/or ameliorating signs of aging on skin of an individual, comprising administering to the individual a composition comprising a compound of formula I.
   In particular, the composition strengthens the DEJ of the skin and promotes cell adhesion and intercellular communication. The composition typically has anti-aging and rejuvenating effects on the skin.
   The cosmetic treatment typically has one or more of the following effects on the skin of an individual:
      Improving skin firmness,
      Increasing volume of the skin,
      Improving skin homogeneity,
      Improving skin complexion,
      Improving skin radiance,
      Increasing skin hydration.
   The cosmetic treatment typically leads to reduction or amelioration of one or more of the following:
      wrinkles,
      thinning,
      decrease of elasticity,
      sagging,
      fine lines,
      hyperkeratosis,
      dry skin,
      loss of skin volume,
      stretch marks,
      sensitivity.
(3) Pharmaceutical composition comprising a compound of formula I for use in treating an age-related skin disease in an individual.

Various non-limiting embodiments relating to the above aspects of the invention are described throughout specification of the invention and illustrated by non-limiting examples.

### FIGURES

FIGS. 1 to 4 show heatmaps using a log 2 scale with upregulation (shown in black) or downregulation (shown in white) of respective proteins upon treatment with 0.01% (01), 0.03% (03) and 0.15% (15) of respective ceramide in vehicle as compared to vehicle without ceramide (VEH) (FIG. 1: CerNS, FIG. 2: LacCerNS, FIG. 3: CerNP, FIG. 4: CerNG); detailed experimental setup explained in Example 1.

FIGS. 5 and 6 show the percentage of surface positive to Laminin 5 (FIG. 5) and Collagen IV (FIG. 6), respectively, immunostaining along the dermal-epidermal junction.

FIG. 7 shows mean concentration of IL-1a released in BEM culture media of non-treated control (T), treated with basis emulsion (E) and treated with basis emulsion with GlcCer (P), respectively, on day 6.

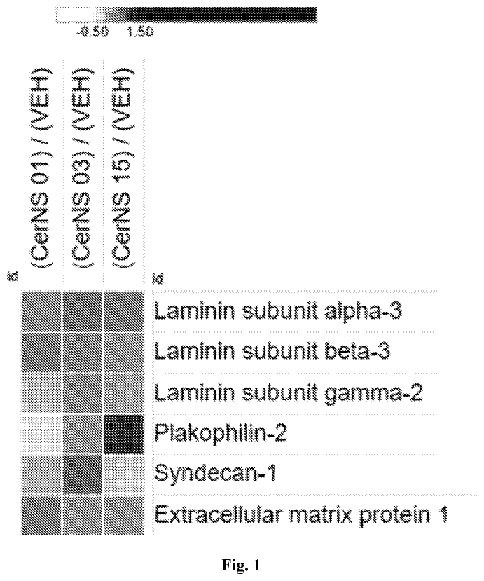

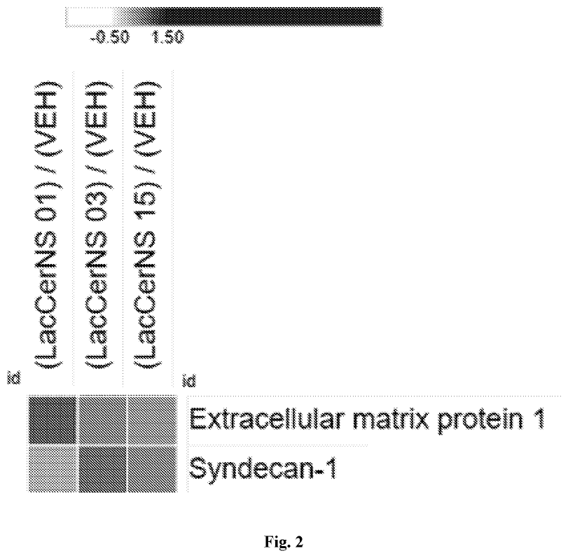

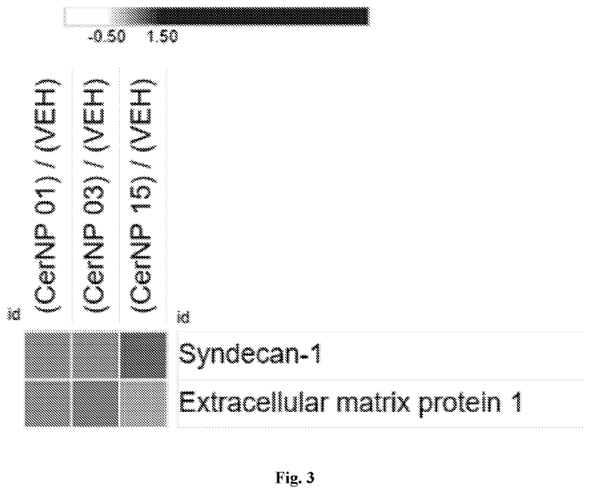

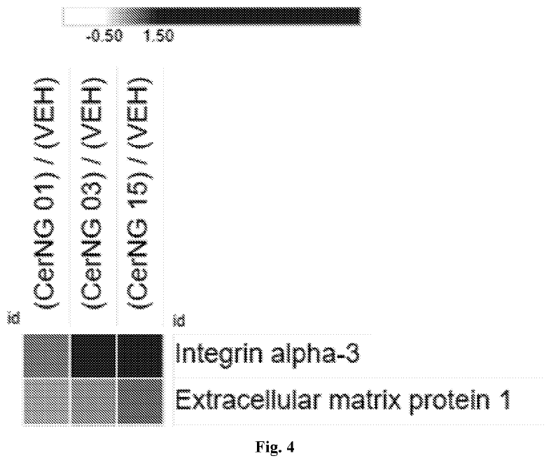

### DETAILED DESCRIPTION OF THE INVENTION

It has now been surprisingly found that compounds of formula I reduce or ameliorate signs of skin aging. In one aspect, the present invention relates to a cosmetic use of a composition for reducing and/or ameliorating signs of skin aging in an individual, wherein the composition comprises a compound of formula I

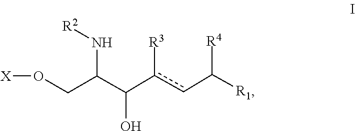

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.

As used herein, the term “alkyl” refers to an acyclic straight hydrocarbyl group in which the carbon atoms may be saturated or contain one or more double and/or triple bonds (so, forming for example an alkenyl or an alkynyl). Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl. Typically, the term alkyl refers to a saturated hydrocarbyl group.

As used herein, the term “acyl” includes both saturated groups and groups having one or more double and/or triple bonds. In the context of acyls, the term “substituted” means that the carbon atoms may be substituted one or several times, preferably 1 to 3 times, with functional group(s). “Skin aging” as used herein refers to both intrinsic and extrinsic skin aging. It includes e.g. inflammaging and photoaging.

“Inflamm-aging” or inflammaging is a chronic, low-grade inflammation that develops with advanced age, in the absence of overt infection, and may contribute to clinical manifestations of other age-related conditions. More precisely, inflamm-aging as used herein denotes skin inflammation as a biological response of a body tissue to both environmental challenges such as sun and chemical pollutants, and internal drivers such as diet, alcohol consumption and smoking, and other potentially harmful stimuli, including pathogens, bacteria, viruses, and other common irritants. Mechanistically, a low-grade, chronic and progressive prominence of pro-inflammatory cytokines over anti-inflammatory cytokines is typically occurring during inflamm-aging.

“Photo-aging” or photoaging refers to the process of chronic sun exposure, including especially exposure to UV-A and UV-B radiation, leading to extrinsic skin aging. The structural and functional manifestations of cutaneous aging are typically premature and more severe in the photo-aged compared to the chronologically aged skin. Photo-aged skin tends to have a rather leathery appearance with deeper wrinkles, laxity, roughness and uneven pigmentation.

“Signs of skin aging” denotes one or more characteristics caused by intrinsic or extrinsic aging processes, such as thin skin, dry skin, fine lines, wrinkles, decreased elasticity and formation of hyperpigmented spots. The signs of skin aging may vary according to various conditions, especially genetic factors. As an example, Asian skin tends to respond differently to sun exposure than Caucasian skin. Asian skin tends to tan more and burn less, which means that chronic UV exposure tends to produce more pigmentation-related problems. Asians recognize melasma, freckles and lentigines as more common phenotypic characteristics of skin aging than fine wrinkles, which appear among the first signs of aging among the Caucasian population.

Cosmetic use for reducing and/or ameliorating signs of skin aging as referred to herein aims at targeting the undesired visual and aesthetic symptoms caused by aging processes, and thereby improve the appearance of the skin.

Signs of skin aging can be measured using clinical and biophysical methods. Functional parameters like skin surface pH, stratum corneum hydration (SCH) or transepidermal water loss (TEWL) are for instance applied as markers of the status and integrity of the skin barrier. During aging, skin surface pH may increase, whereas TEWL decreases. Structural assessments such as skin microtopography and Cutometer® measurements may be used to measure surface roughness and skin elasticity respectively. During aging, surface roughness may increase due to a decrease of isotropy, whereas skin elasticity decreases. Age-dependent skin colour changes, such as the degree of luminance and yellowness in the skin tone, may be measured using spectrophotometry and expressed as L* and b* respectively in the CIE L*a* b* colour space. In addition to these instrumental measures, clinical skin aging scales are used in skin research and aesthetic dermatology. These scales are generally based on skilled assessment of skin dyspigmentation, wrinkling and sagging. Assessment is performed on standardized photographs taken from the test subject's face from a front and 450 right and 450 left view.

The term “reducing” or any variation of the term such as reduction includes any measurable decrease to achieve a desired effect, to restore a previous state or to weaken an undesired effect. In the context of “reducing signs of skin aging” on skin, the term reducing is to be understood as reduction of formation of undesired visual and aesthetic signs. The term ranges from preventing to reducing and/or slowing of the formation of signs of skin aging.

“Ameliorating signs of skin aging” refers to the counteraction against signs of skin aging that have been caused by the respective aging process. For example, it refers to reducing size, depth and amount of wrinkles that are already present on the skin.

“Treatment” or any variation of the term such as treating means to address a condition with the objective of improving or stabilising an outcome in the individual being treated or addressing an underlying need. Treating therefore includes the management of the condition or dermatological needs of the individual being treated. Treating can include cosmetic or medical treatment. Treatment can include preventive or curative treatment. Preventive treatment can include primary prevention or secondary prevention. Cosmetic treatment means to address undesired visual signs of the skin, without any therapeutic effects.

According to the invention, treatment using compositions of the invention may be either topical or oral. By “topical” is meant that a composition is administered on the surface of keratinous tissue, especially the skin. Such composition is denoted herein as “topical composition”. By “oral” is meant that a composition is administered to a subject-in-need orally, i.e. through the subject mouth. Such composition is denoted herein as “oral composition”.

The compound of formula I may be obtained by any technique known to the skilled person, such as by a fermentation technique or by a biocatalytic process. For example, lactosylceramide may be produced starting from lactosyl fluoride, followed by coupling to an acylated sphingoid base by the use of an endoglycoceramide synthase, such as described in Rich et al., ChemComm (2011) 47:10806-10808. Sphingoid bases may be produced by a fermentation process, such as e.g. described in WO 95/12683 A1, especially phytosphingosine or a precursor thereof (claim 1 of EP 0 726 960 B1).

In preferred embodiments, R¹ of the compound of formula I is −C₁₂H₂₅.

In preferred embodiments, R² of compound I is an unsubstituted acyl group. More preferably, R² of compound I is a saturated fatty acyl group or a fatty acyl group having one or more double bonds, including especially hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl and 30-(linoleyloxy)-triacontanoyl.

In some preferred embodiments, R¹ of the compound of formula I is —C₁₂H₂₅ and R² is octadecanoyl.

The compound of formula I may be a ceramide or a glycoceramide.

In the context of the invention it has been found that application ceramide NS (Cer NS), of lactosyl ceramide (LacCer), of ceramide NP (Cer NP) and of ceramide NG (Cer NG) on keratinocytes leads to an up-regulation of structural proteins of the skin and/or of proteins involved in cell adhesion or cellular communication. Accordingly, preferred ceramides are Cer NS, Cer NP and Cer NG. A preferred glycoceramide is LacCer.

Ceramides as used herein are named according to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. For each species, the number of carbons and unsaturations (if present) may be expressed in parentheses following the letters of N, A, E, and O. When not specified, the carbon chain length of the sphingoid base moiety of the ceramides mentioned herein is typically Cis. When not specified, the carbon chain length of the acyl moiety of the ceramides mentioned herein may be e.g. C₁₆, C₁₇, C₁₈, C₁₉, C₂₀, C₂₂, C₂₄, C₂₆, C₂₈, C₃₀, C₃₂ or C₃₄.

Accordingly, Cer NS refers to a ceramide comprising a non-hydroxy acid as acyl moiety and sphingosine as sphingoid base moiety. Typically, both carbon chain lengths are C₁₈. Cer NP refers to a ceramide comprising a non-hydroxy acid as acyl moiety and phytosphingosine as sphingoid base moiety. Typically, both carbon chain lengths are C₁₈. Cer NG refers to a ceramide comprising a non-hydroxy acid as acyl moiety and dihydrosphingosine as sphingoid base moiety. Typically, both carbon chain lengths are Cis. Ceramide NG may also be referred to as ceramide NdS and can be used herein interchangeably.

LacCER as mentioned herein is preferably ceramide NS and has an acyl chain length of Cis. Lactosyl ceramide NS with an acyl chain length of C₁₈ may be referred to herein also as LacCNS. GlcCER as mentioned herein is preferably ceramide NS and has an acyl chain length of Cis. Glucosyl ceramide NS with an acyl chain length of C₁₈ may be referred to herein also as GlcCNS.

The composition comprising the compound of formula I may, in some embodiments, be a topical composition or, in other embodiments, an oral composition.

An oral composition may be in solid or liquid form, such as a tablet, a capsule, a powder, a syrup, an emulsion, or the like. The oral composition may be in form of a nutritional composition, such as a food composition or a nutritional supplement. The skilled person will be aware of suitable formulations that enable a safe delivery of an effective amount of the composition.

Generally, oral compositions of the present invention will contain from 1 mg to about 500 mg of a compound of formula I, such as from about 1 mg to about 100 mg of a compound of formula I, or from about 5 mg to about 200 mg of a compound of formula I. For example, the oral compositions of the present invention may contain from about 1 mg to about 10 mg of a compound of formula I, or from about 5 mg to about 5 mg of a compound of formula I.

A “topical composition” as used herein includes compositions suitable for topical application on keratinous tissue, especially on skin. Such compositions are typically dermatologically acceptable in that they do not have undue toxicity, incompatibility, instability, allergic response, and the like, when applied to skin. Topical compositions typically comprise a topically acceptable carrier. Topical skin care compositions of this invention can have a selected viscosity to avoid significant dripping or pooling after application to skin. Accordingly, a topical composition of the invention comprising one or more compounds of formula I, would typically comprise one or more other compounds that are useful for formulating these compositions. Typically, such “formulating” compounds do not possess the biological activity of the compounds of the invention.

Keratinous tissue includes keratin-containing layers disposed as the outermost protective covering of mammals and includes, but is not limited to, lips, skin, scalp, hair and nails.

Skin cells may refer herein to any skin resident cell type, including e.g. dermal fibroblasts, melanocytes and epidermal keratinocytes, or cell types from skin appendages, such as sebocytes, or a mixture thereof.

Generally, topical compositions of the present invention will contain from about 0,0001% to about 10% wt/wt of a compound of formula I, such as from about 0,005% to about 5% wt/wt of a compound of formula I, or from 0.01% to about 1% wt/wt of a compound of formula I. For example, the topical compositions of the present invention may contain from about 0,0001 to about 0,00025% wt/wt of a compound of formula I, from about 0,0002 to about 0,0004% wt/wt of a compound of formula I, from about 0,0003 to about 0,0005% wt/wt of a compound of formula I, from about 0,0004 to about 0,0006% wt/wt of a compound of formula I, from about 0,0005 to about 0,0008% wt/wt of a compound of formula I, from about 0,0007 to about 0.001% wt/wt of a compound of formula I, from about 0.001 to about 0,005% wt/wt of a compound of formula I, from about 0,003 to about 0,008% wt/wt of a compound of formula I, from about 0,005 to about 0.01% wt/wt of a compound of formula I, from about 0,008 to about 0.05% wt/wt of a compound of formula I, from about 0.05 to about 0.08% wt/wt of a compound of formula I, from about 0.06 to about 0.1% wt/wt of a compound of formula I, from about 0.08 to about 0.1% wt/wt of a compound of formula I, etc.

In some embodiments, topical compositions of the present invention contain an amount of about 0.01% to 5% wt/wt of a compound of formula I, or an amount of about 0.02% wt/wt to 1% wt/wt of a compound of formula I. Examples are 0.05% wt/wt, 0.1% wt/wt or 0.2% wt/wt of a compound of formula I.

The terms “about” or “approximately” are defined as being close to the value or range following these terms, as understood by one of ordinary skill in the art, and include a deviation of up to 10% of the values or ranges that follow.

The composition may in some embodiments comprise more than one compound of formula I, also termed herein as blend of compounds of formula I. In such blend compositions, each compound of formula I may be present in the same amount or in different amounts. The ranges or amounts as listed above may relate to the total content of the blend in the composition, or to the amount of one compound in the blend in the composition.

The composition according to the present invention may have a pH in the range of about 3 to about 8, e.g. from about 3 to 5 or from about 4.5 to 6.

The composition according to the present invention is used to reduce and/or ameliorate signs of skin aging. In particular, the composition according to the present invention is used to improve the biomechanical properties of the skin. In preferred embodiments, the composition according to the present invention is used to improve skin firmness, to increase skin volume, to improve skin homogeneity, to improve skin complexion, to improve skin radiance and/or to increase skin hydration, especially to reduce or ameliorate wrinkles, thinning, decrease of elasticity, sagging, fine lines, hyperkeratosis, dry skin, loss of skin volume, stretch marks and/or sensitivity.

In some embodiments, the invention relates to a non-therapeutic method for reducing signs of aging on skin of an individual, the method comprising administering to the individual an effective amount of a composition comprising a compound of formula I.

In some embodiments, the invention relates to a non-therapeutic method for improving skin firmness in an individual, the method comprising administering to the individual an effective amount of a composition comprising a compound of formula I.

In some embodiments, the invention relates to a non-therapeutic method for increasing skin volume in an individual, the method comprising administering to the individual an effective amount of a composition comprising a compound of formula I.

In some embodiments, the invention relates to a non-therapeutic method for improving skin homogeneity in an individual, the method comprising administering to the individual an effective amount of a composition comprising a compound of formula I.

In some embodiments, the invention relates to a non-therapeutic method for improving skin complexion in an individual, the method comprising administering to the individual an effective amount of a composition comprising a compound of formula I.

In some embodiments, the invention relates to a non-therapeutic method for improving skin radiance in an individual, the method comprising administering to the individual an effective amount of a composition comprising a compound of formula I.

In some embodiments, the invention relates to a non-therapeutic method for increasing skin hydration in an individual, the method comprising administering to the individual an effective amount of a composition comprising a compound of formula I.

An “effective amount” means an amount sufficient to render a desired treatment or management outcome in an individual. An effective amount can be administered in one or more doses, typically in more doses, to achieve the desired treatment or management outcome. The effective amount may vary and depend on the type of administration, e.g. whether it is an oral or a topical administration. The effective amount of a compound of formula I in a composition of the invention may further vary depending upon factors such as the age of the person, the risk and severity of any underlying condition, the environment the person is exposed to, the form of the composition, its use for cosmetic or medical purposes, and any other skin care compositions being administered. Appropriate doses for any particular person or indication may be determined by methods known to skin care practitioners. The dose may also vary depending upon whether the topical composition is a “leave on” or “rinse off” formulation with “rinse off” formulations generally containing lower doses. Further, the dose may vary depending on whether the composition is applied in an intervention treatment phase or in a maintenance phase. Generally higher doses can be applied in an initial intervention phase than in a maintenance phase.

Biomechanical properties of the skin refer to the physiological properties of the epidermis and dermis that provide protection against biological invasion, injury and UV radiation and resist the loss of skin integrity that occurs with movement, stretching and application of force.

Skin firming relates to the improvement of elasticity and DEJ; making the skin stronger and more resilient by maintaining or increasing its elasticity.

Skin elasticity refers to a property of the skin that enables it to change and recover shape when stretched or deformed. Loss of skin elasticity or elastosis causes the skin to be saggy, crinkled or leathery.

Volume of the skin refers to the volume or capacity of the skin layers. Skin with increased volume looks lifted, healthy and young.

Skin homogeneity denotes the evenness of the reflectance properties of the skin.

An improvement of skin complexion as used herein refers to the colour and texture of the skin, especially on the face. A disrupted skin barrier may result in a loss of skin complexion.

Improving skin radiance as used herein refers to skin tone, luminosity, firmness, and discolouration (thin dark circles and sun damage).

Wrinkles as used herein relates to, in particular, the face skin wrinkles, both fine surface lines and deeper furrows.

Skin pigmentation refers to the amount and type of melanin, a pigment made by specialized skin cells, the melanocytes. Skin pigmentation is determined by the balance of two types of melanin in the skin, eumelanin and pheomelanin, which are characterized by different colors and functions. This balance can shift depending on hormones, interactions with other cells or genetic factors. Changes in melanin production can cause pigment disorders, such as hyperpigmentation (dark spots), hypopigmentation (light spots) or depigmentation (white spots or patches) Skin damage from acne, blisters, cuts, sun exposure, genetic factors and autoimmune conditions are possible causes for changes in melanin and skin pigmentation.

Thin skin as referred to herein is a result of thinning of the outermost layer of the skin, the epidermis, and/or of a flattened epidermal-dermal junction and accordingly a less abundant extracellular matrix. It is more common in older adults and is most visible on the face, arms, and hands. The veins, tendons, bones, and capillaries under the skin of thin hands and arm may be visible to some extent.

Hyperkeratosis denotes an overgrowth and thickening of the outermost layer of the epidermis, the stratum corneum.

Stretch marks denote irregular lines or streaks on the skin where it has been stretched or distended, especially due to pregnancy or obesity.

Sensitive skin can be considered as a condition wherein the skin exhibits a reduced tolerance to stimuli that normally should not provoke such sensations. Such stimuli can include e.g. environmental stresses such as cold or wind exposure, dryness, (tobacco) smoke, irritant side effects of medicaments or cosmetics, or the like. A disrupted skin barrier may increase skin sensitivity.

In the context of the invention it has been found that application of Cer NS, LacCer, Cer NP and Cer NG on keratinocytes leads to an up-regulation of structural proteins of the DEJ and of markers involved in cell adhesion and cellular communication. More precisely, the application of Cer NS leads to up-regulation of ECM1, of Syndecan-1, of Laminin-5 and of Plakophilin-2. LacCer and Cer NP lead to up-regulation of ECM1 and of Syndecan-1. Cer NG leads to up-regulation of ECM1 and of Integrin alpha-3.

In some embodiments, Cer NS is used for up-regulating ECM1.

In some embodiments, Cer NS is used for up-regulating Syndecan-1.

In some embodiments, Cer NS is used for up-regulating Laminin-5.

In some embodiments, Cer NS is used for up-regulating Plakophilin-2.

In some embodiments, LacCer is used for up-regulating ECM1.

In some embodiments, LacCer is used for up-regulating Syndecan-1.

In some embodiments, Cer NP is used for up-regulating ECM1.

In some embodiments, Cer NP is used for up-regulating Syndecan-1.

In some embodiments, Cer NG is used for up-regulating ECM1.

In some embodiments, Cer NG is used for up-regulating Integrin alpha-3.

Syndecan-1, ECM1, Laminin-5 and Integrin a mediate matrix adhesion and can bind several structural proteins of the DEJ, which help maintaining a functional dermal-epidermal interface. They decline during aging, leading to a weakened interface with flattened junctions. This leads to signs of aging, including wrinkle formation and reduced elasticity.

ECM1 plays a pivotal role in the structural and homeostatic biology of the skin, particularly in the proliferation and differentiation of epidermal keratinocytes, reconstitution of basement membrane and aging. ECM1 furthermore acts as an inhibitor of matrix metalloprotease 9 (MMP9), a protease that degrades several structural elements of the ECM like collagen, but also directly targets Syndecan1. MMP 9 is activated by oxidative stress and the resulting formation of reactive oxygen species (ROS) from external stressors like UV radiation and pollution, thereby accelerating the aging effects. ECM1 can bind to fibronectin, type IV collagen and laminin 332. Type IV collagen can be used herein interchangeably with collagen IV. Laminin-5 can be used herein interchangeably with Laminin-332. Laminin-332 is a major hemidesmosomal component of the skin basement membrane. It functions as a ligand for α3β1 and α6β4 integrins to regulate adhesion, migration, and morphogenesis of basal keratinocytes. Integrins are the main adhesion proteins that bridge the cellular cytoskeleton with the ECM and thereby serve as bidirectional signal transducers regulating cell proliferation, homeostasis, differentiation, adhesion, migration and apoptosis. The integrin family consists of genetically distinct alpha and beta subunits that heterodimerize to form functioning transmembrane receptors

Syndecans belong to the family of proteoglycans, which are the fundamental component of the extracellular matrix; therefore, they are the main substance that fills up the spaces existing between the organism's cells. Here, they form large complexes, both with other proteoglycans and with hyaluronic acid and proteins such as collagen, maintaining the skin's firmness and preventing the formation of wrinkles.

Plakophilins are important for cell adhesion, cell-cell communication by enhancing desmosomal stability by binding other desmosomal components. They are localized at the cytoplasmic face of desmosomes where they participate in linking the intermediate filament cytoskeleton to the junctional plaque. Plakophilin-2 is a constitutive component of desmosomes at sites of cell-cell contact within epithelial cells.

Without being bound to theory, it is believed that Cer NS, LacCer, Cer NP and Cer NG have anti-aging effects by counteracting the weakening of the DEJ and decline of cell adhesion and cellular communication, processes that occur during aging. As a consequence, a limitation of signs of aging, such as wrinkle formation or loss of elasticity and sagging is expected.

Providing mechanical stability to the skin, particularly strengthening the DEJ structure and promoting cell adhesion and cellular communication, as described herein, is an efficient way of combating signs of skin aging. However, Cer NS, LacCer, Cer NP and Cer NG can modulate other mechanisms which strengthen the skin or reduce aging effects on the skin. Also, there are many negative effects of aging on the skin and its appearance and it is expected that a reduction of skin aging will reduce these other undesired effects.

In this context, it has been further found that application of GlcCer, LacCer and Cer NG on keratinocytes leads to an up-regulation of proteins of the mitochondrial respiratory chain and associated with the generation of ATP. More precisely, the application of GlcCer leads to up-regulation of ATP synthase subunit g (mitochondrial), of ATP synthase subunit delta (mitochondrial), of cytochrome c oxidase subunit 6C, of NADH dehydrogenase [ubiquinone] iron-sulfur protein 2 (mitochondrial), of NADH dehydrogenase [ubiquinone]1 alpha subcomplex subunit 8, of ATP-dependent zinc metalloprotease YME1L1, of NAD-dependent malic enzyme (mitochondrial) and of phosphomevalonate kinase. LacCer leads to an upregulation of ATP synthase subunit g (mitochondrial), of ATP synthase subunit delta (mitochondrial), of cytochrome c oxidase subunit 6C, of cytochrome c oxidase subunit 2, of cytochrome c oxidase subunit NDUFA4, of cytochrome b-cl complex subunit 9, of ATP-dependent zinc metalloprotease YME1L1, of NAD-dependent malic enzyme (mitochondrial) and of phosphomevalonate kinase. Cer NG leads to an upregulation of ATP synthase subunit g (mitochondrial), of ATP synthase subunit delta (mitochondrial), of cytochrome c oxidase subunit 6C and of phosphomevalonate kinase.

The skin has a continuously regenerating epidermis. The epidermal progenitors are thus highly proliferative and metabolically active, for which much energy is required. The energy requirements are met by mitochondrial respiration, an adenosine triphosphate (ATP) generating process. Activating the respiratory chain and therefore ATP generation keeps the metabolism high and provides energy for repair mechanisms. Furthermore, an activated metabolism supports skin regeneration and helps to fight against signs of ageing.

Without being bound to theory, it is expected that an up-regulation of these markers leads to an increase in mitochondrial ATP production and consequently a boost in cell energy. In turn, adverse phenotypic manifestations caused by mitochondrial dysfunctions, including signs of ageing, especially inflamm-aging and photo-aging, which further impact the healthy look of the skin, would be prevented or reduced.

It has been also further found that application of LacCer, Cer NS and Cer NG on keratinocytes leads to an up-regulation of enzymes involved in oxidative stress defence and in maintaining cell function homeostasis. More precisely, the application of LacCer leads to up-regulation of Glutathione S-transferase Mu 2, cystathione-gamma-lyase, NAD-dependent protein deacetylase sirtuin-2 (sirtuin-2), LC3B (microtubule-associated proteins 1A/1B light chain 3B), Syndecan-1 and Extracellular Matrix Protein 1 (ECM1). The application of Cer NS leads to an up-regulation of cornifin-B and sirtuin-2. The application of Cer NG leads to an up-regulation of small proline-rich protein 2D and sirtuin-2.

External influences including UVR and air pollutants lead to an increase in the formation of free radicals in skin, most notably of reactive oxygen species (ROS). The formation of free radicals is a pivotal mechanism leading to skin aging. These external influences can result in greater production of ROS than the endogenous defence mechanisms are able to deal with. Especially during aging, the endogenous defence mechanisms that counter ROS become less effective. The resulting imbalance leads to the progressive oxidative damage of cellular structures, which accelerates aging processes and signs of oxidative stress.

Without being bound to theory, it is expected that up-regulation of these markers leads to an enhanced oxidative stress response and to a better defence against pollutants and aging-related mechanisms in the skin. As a consequence, a protection or reduction of oxidative stress-related signs and effects on skin are expected.

It has been additionally found that application of LacCer, GlcCer and Cer NS on keratinocytes leads to an up-regulation of proteins involved in autophagy and in maintaining cell function homeostasis. More precisely, the application of LacCer leads to up-regulation of LC3B (microtubule-associated proteins 1A/1B light chain 3B), and GlcCer and Cer NS lead to up-regulation of LC3A (microtubule-associated proteins 1A/1B light chain 3A).

Beyond its function in the adaptation of individual cells or organs to changing conditions, autophagy interacts with aging processes. Whereas autophagy decreases the rate of aging, the activity of autophagy declines during aging. Based on that interplay, stimulation of autophagy is considered to have potent anti-aging effects.

Without being bound to theory, it is believed that LacCer, GlcCer and Cer NS are involved in the activation of autophagy and mitophagy and therefore can play a role in cell detoxification and recycling and promoting cell and tissue longevity. As a consequence, a limitation of signs of age is expected.

Further, it has been found that application of GlcCer and of Cer NS on keratinocytes leads to a down-regulation of pro-inflammatory markers. More precisely, the application of GlcCer leads to a down-regulation of pro-inflammatory regulators, in particular cytokines and proteins of the S100 family, especially interleukin-1-alpha (IL-1α), protein S100-A2, protein S100-A9, and nuclear factor NF-kappa-B p105 subunit. The application of Cer NS leads to a down-regulation of proteins of the S100 family, especially protein S100-A2 and protein S100-A9.

Aging skin experiences chronic inflammation that naturally occurs also in the absence of overt infection, a phenomenon described as inflamm-aging. One major factor contributing to inflamm-aging is the excessive production of mitochondrial reactive oxygen species (ROS). Increased levels of ROS in turn activate inflammatory signalling pathways leading to a sustained state of chronic inflammation.

Without being bound to theory, it is expected that down-regulation of these markers leads to a reduction in inflammatory processes and consequently a prevention of undesired signs and effects of inflammation on skin.

It has also been further found that application of Cer NP on keratinocytes leads to an up-regulation of Syndecan-1 and Keratin-17, which are involved in tissue repair. Tissue repair is critical for survival of all organisms. Skin wound healing occurs through a dynamic and complex process. Perturbation of the tightly regulated interactions slows down the repair response and impairs the quality of healing. Impaired healing leads to chronic wounds, infection and pathological scarring. Aging is accompanied by a gradual decline of tissue integrity, function and regenerative capacity.

Without being bound to theory, it is expected that up-regulation of these markers leads to an improvement of closure of a wound, enhanced cell migration and proliferation and thereby reduces effects of aging skin.

It has been further found that application of Cer NS on keratinocytes leads to an up-regulation of Kallikrein-5, a protease that cleaves corneodesmosomes, which are involved in intracellular adhesion of corneocytes, which allows desquamation. Skin desquamation, also termed as skin peeling, describes the last step of epidermal differentiation, wherein dead corneocytes are removed and skin renewal in the lower layers can be triggered. In aging skin the process of desquamation becomes less effective. Without being bound to theory, it is expected that up-regulation of Kallikrein-5 leads to an enhancement of desquamation and thereby counteracts effects of aging skin.

In the context of the present invention it has been further found that IL-1a release is increased in skin explants upon GlcCer application.

IL-1a is best known for its role in the regulation of the immune response. Due to its pleiotropic nature, it has, however, other functions. In skin, IL1a acts as a messenger regulating skin homeostasis, and it is highly and constitutively expressed by keratinocytes in the epidermis. The epidermis, in particular the stratum corneum, is the tissue which contains the highest content of IL-1a in the human body. Keratinocyte-derived IL-1a induces fibroblasts to express growth factors which act back on the epidermis and stimulate its regeneration. The expression of IL-1a by keratinocytes and the production of collagen have been shown to decline in aging skin. The resulting deficit of IL-1a has been hypothesized to be at least partially responsible for the signs of skin aging. Without being bound to theory, it is believed that GlcCer has anti-aging properties, by e.g. improving skin elasticity and increasing skin density, when applied on skin and therefore can reduce signs of skin aging.

In some embodiments, the invention relates to a method for increasing IL-1a levels in the epidermis, the method comprising administering to the individual an effective amount of a composition comprising a compound of formula I.

The composition according to the present invention may also include additional ingredients typically found in cosmetic and skin care formulations such as cosmetic ingredients.

In some embodiments, the composition may advantageously further comprise hyaluronic acid, alpha-hydroxy acids (AHA), polyhydroxy acids (PHA), a peptide imitating a sequence of a structural protein such as collagen or elastin, or an anti-oxidant, including e.g. melatonin, a vitamin, a carotenoid, a polyphenol, coenzyme Q10 or azelaic acid.

Peptides (e.g. polypeptides or oligopeptides) are composed of amino acids and can imitate a peptide sequence, typically a functional sequence, of molecules such as collagen or elastin. Through topical application, polypeptides have the ability to stimulate collagen synthesis and activate dermal metabolism.

Vitamins may be e.g. vitamin C, vitamin B3, vitamin A, or vitamin E. The term vitamin as used herein includes sources, metabolites or derivatives thereof, for example vitamin C may also be in the form of ascorbyl palmitate, ascorbyl glucoside, magnesium ascorbyl phosphate, ascorbyl lysostearate, vitamin A may be vitamin A palmitate (retinyl palmitate) and vitamin E may be vitamin E linoleate (tocopheryl linoleate). Other esters of vitamin C, vitamin A and vitamin E may also be utilised.

Carotenoids include e.g. carotene, curcumin or astaxanthin. Polyphenols include e.g. resveratrol, phenolic acid, aloesin, lignan, aloesin, arbutin, camosic acid, epigallocatechin-3-gallate or anthocyanin.

Other cosmetic ingredients that may be comprised in especially a topical composition include niacinamide, bisabolol, colloidal oatmeal, zinc pyrrolidone carboxylic acid (PCA), panthenol, allantoin, aloe vera, sea buckthorn oil, chamomile, cucumber extract, feverfew, ginseng, turmeric, selenium, sea buckthorn oil, calendula oil, licorice extract, coal tar solution, evening primrose oil, borage oil, luteolin, myricetin, dihydroeugenol, kaempherol, caffeic acid, epigallocatechin gallate, or a combination thereof.

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and colour ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavouring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellents, UV absorbers and/or reflectors (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminium starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminium hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate).

The topical composition of the present invention typically include or are incorporated into different types of vehicles and carriers. In some embodiments, the topical composition of the present invention further comprises a topically acceptable carrier. The vehicle or carrier can be a dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

Topical compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydroalcoholic), anhydrous bases (such as lipsticks and powders), gels, masks, peelings, and ointments. Variations and other structures will be apparent to the skilled artisan and are appropriate for use in the present invention.

The topical composition of the present invention is typically intended for application on a specific area of the skin, such as on the face or an area of the face, on the body or an area of the body and/or on the hair or scalp. The topical composition preferably is for application on the face and/or on the body. A specific area of the face is e.g. the area around the eyes. A specific area on the body includes e.g. the hands, the arms or the legs.

In preferred embodiments, the topical composition is formulated as an emulsion, an oil, a toner or a gel and for application on the face and/or on the body.

The inventive use of the composition in general is intended an individual who is a mammal. Especially, the individual is a human. In some preferred embodiments, the human individual is an adult individual, i.e. a human of 20+ years old, especially 30+ years old, such as 40+ years old or 50+ years old; in other preferred embodiments, the human individual may be a child or a teenager.

The individual may in some embodiments have an exposure to an environmental stressor. The exposure may be before the use of the composition of the invention, during the use of the composition of the invention and/or in the planned future relative to the use of the composition of the invention. The skilled person will understand that the term exposure typically relates to a prolonged exposure, such as for a year or many years.

Main environmental stressors having an impact on the skin are solar radiation, especially ultraviolet (UV) light, polycyclic aromatic hydrocarbons (PAHs), volatile organic compounds (VOCs), ozone, nitrogen and sulphur oxides, particulate matter PM2.5, blue light, ozone, or tobacco smoke. Particulate matter (PM) is divided according to particle size: PM10 (coarse particles), PM2.5 (fine particles) and PM0.1 (ultra-fine particles), with a respective diameter of below 10, 2.5 and 0.1 μm.

Depending on the type of environmental stressor, some body parts are typically more affected than others, and are therefore more preferred for the inventive use. For example, the face, hands and maybe arms and legs are more exposed to UV radiation than the rest of the body and topical cosmetic could be adapted accordingly.

The individual may in some embodiments live an unhealthy lifestyle. The unhealthy lifestyle may be before the use of the composition of the invention, during the use of the composition of the invention and/or in the planned future relative to the use of the composition of the invention. The skilled person will understand that the unhealthy lifestyle typically relates to a prolonged unhealthy lifestyle, such as for a year or many years.

An unhealthy lifestyle may include lack of sleep, smoking, alcohol abuse, unhealthy diet, or a combination thereof.

The individual may in some embodiments have heredity of premature skin aging. For example, the individual may have a biological family in which early and strong wrinkle formation or sensitive skin is common. Premature skin ageing may occur due to intrinsic and extrinsic factors, like e.g. genetic factors causing early signs of aged skin, or an unhealthy eating or lifestyle such as increased UV light exposure.

In some embodiments, the compound of formula I is incorporated into a pharmaceutical composition that may be used for treating an age-related skin disease in an individual. Age-related skin diseases include especially skin cancer, such as squamous cell carcinoma or melanoma.

In some embodiments, the compound of formula I is a pharmaceutically non-active ingredient in the pharmaceutical composition. It may then act as an enhancer of the pharmaceutically active ingredient of the pharmaceutical composition, or it may cosmetically treat visual effects that the skin disease causes. The pharmaceutically active ingredient of the pharmaceutical composition may e.g. be a hormone, such as estradiol or progresterone.

In other embodiments, the compound of formula I is pharmaceutically active for treating the autophagy-related skin disease.

In some embodiments, R¹ of the compound of formula I of the pharmaceutical composition is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, 30-(linoleyloxy)-triacontanoyl.

The pharmaceutical composition may comprise a further ingredient that is pharmaceutically active for treating an autophagy-related skin disease and is structurally different from a compound of formula I.

The compositions of the invention may be advantageously administered to an individual for at least 7 days, such as for at least 14 days or for at least 21 days. For example, the composition is administered for at least 28 days. In practice, there is no time limitation for the administration period of a cosmetic composition of the invention. For pharmaceutical composition of the invention, the administration period would be typically determined by a medical practitioner for each individual patient.

A topical or oral composition (either cosmetic or pharmaceutical) may be administered one time or several times per day, such as 2-3 times per day. Typically, the composition is administered for consecutive days.

Different aspects of the invention discussed above are described in the following selected, but not limiting, embodiments:
1. Cosmetic use of a composition for reducing and/or ameliorating signs of skin aging in an individual, wherein the composition comprises a compound of formula I

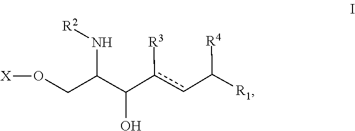

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.
2. Cosmetic use according to embodiment 1, wherein the composition is a topical or an oral composition.
3. Cosmetic use according to embodiment 1 or 2, wherein R¹ of the compound of formula I is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, and 30-(linoleyloxy)-triacontanoyl.
4. Cosmetic use according to any one of embodiments 1 to 3, wherein the compound of formula I is present in a topical composition in an amount of about 0.0001% to about 10% wt/wt, such as from about 0.005% to about 5% wt/wt, or from about 0.05% to about 0.2% wt/wt.
5. Cosmetic use according to any one of embodiments 1 to 4, wherein the composition comprises
   a compound of formula I wherein X is lactosyl and R² is a substituted or unsubstituted acyl having 16-34 carbon atoms,
   a compound of formula I wherein the carbon-carbon bond noted [symbol] is a single bond, X is hydrogen, R¹ is —C₁₂H₂₅, R² is an unsubstituted acyl having 16-34 carbon atoms, and R³ is a hydroxyl group,
   a compound of formula I wherein the carbon-carbon bond noted [symbol] is a single bond, X is hydrogen, R¹ is —C₁₂H₂₅, R² is an unsubstituted acyl having 16-34 carbon atoms, and R³ is hydrogen, and/or
   a compound of formula I wherein the carbon-carbon bond noted [symbol] is a double bond, X is hydrogen, R¹ is —C₁₂H₂s and R² is an unsubstituted acyl having 16-34 carbon atoms.
6. Cosmetic use according to any one of embodiments 1 to 6, comprising an increase of expression levels of one or more structural proteins of the skin, especially of the dermal-epidermal junction (DEJ), such as ECM1, Syndecan-1, Integrin alpha-3 or Laminin-5, and/or of proteins involved in cell adhesion or cellular communication, such as Plakophilin-2.
7. Cosmetic use according to any one of embodiments 1 to 6, wherein reducing and/or ameliorating signs of skin aging comprises strengthening the skin structure, especially the structure of the dermal-epidermal junction (DEJ) and/or promoting cell adhesion and communication.
8. Cosmetic use according to any one of embodiments 1 to 7, having one or more of the following effects on the skin of an individual:
   Improving skin firmness,
   Increasing volume of the skin,
   Improving skin homogeneity,
   Improving skin complexion,
   Improving skin radiance,
   Increasing skin hydration.
9. Cosmetic use according to any one of embodiments 1 to 8, wherein reducing and/or ameliorating signs of skin aging comprises reducing or ameliorating one or more of the following signs on the skin:
   wrinkles,
   thinning,
   decrease of elasticity,
   sagging,
   fine lines,
   hyperkeratosis,
   dry skin,
   loss of skin volume,
   stretch marks,
   sensitivity.
10. Method for reducing and/or ameliorating signs of aging on skin of an individual, the method comprising administering to the individual a composition comprising a compound of formula I

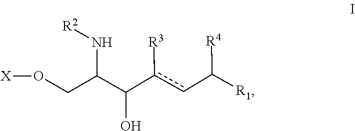

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.
11. Cosmetic use according to any one of embodiments 1 to 9 or method according to embodiment 10, wherein the compound of formula I is an anti-aging compound.
12. Cosmetic use or method according to any one of embodiments 1 to 11, wherein the composition comprises a further agent for reducing or ameliorating skin aging, such as hyaluronic acid, melatonin, a peptide imitating a functional sequence of a structural protein such as collagen or elastin, or an anti-oxidant, including a vitamin, a carotenoid, a polyphenol, coenzyme Q10 or azelaic acid.
13. Cosmetic use or method according to any one of embodiments 1 to 12, wherein the individual is a human individual.
14. Cosmetic use or method according to any one of embodiments 1 to 13, wherein the individual has one or more of the following:
   exposure to an environmental stressor,
   heredity of premature skin aging,
   an unhealthy lifestyle.
15. Cosmetic use or method according to embodiment 14, wherein the environmental stressor includes climatic conditions, such as UV light, or chemical pollutants, such as polycyclic aromatic pollutants (PAH), particulate matter PM2.5, blue light, ozone, or cigarette smoke.
16. Cosmetic use or method according to embodiment 14 or 15, wherein the unhealthy lifestyle includes lack of sleep, smoking, alcohol abuse, unhealthy diet, or a combination thereof.
17. Cosmetic use or method according to any one of embodiments 14 to 16, wherein the exposure to the environmental stressor and the unhealthy lifestyle include a prolonged past, present and/or future exposure, such as for around 1 month, preferably for a longer period of time, e.g. around 3-6 months, around 1 year, 5 years, 10 years, 20 years or longer.
18. Cosmetic use or method according to any one of embodiments 1 to 17, wherein the composition is administered to an individual for at least 2 days, especially for at least 7 days or for at least 14 days, such as for 28 days.
19. Pharmaceutical composition comprising a compound of formula I,

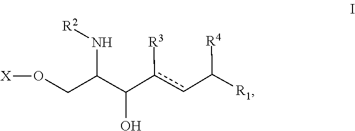

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety, for use in treating an age-related skin disease in an individual, such as skin cancer.
20. Pharmaceutical composition according to embodiment 19, further comprising a compound that is
   structurally different from a compound of formula I and
   pharmaceutically active for treating the skin disease.

### EXAMPLES

The examples described below are for illustration purposes only. The invention is not limited in scope to embodiments described in the examples.

### Example 1: Characterisation of Effects of (Glyco)Ceramides as Cosmetic Active Ingredients

Material and methods: Reconstructed Human Epidermis (RHE) cell culture is used, as this system mimics the in vivo 3D structure of epidermal tissue as well as the conditions and processes that occur in normal epidermis. RHE are cultured using the medium provided by the manufacturer until analysis. RHE are maintained in survival for 24 hours, before RHE are topically treated with the respective ceramide in a base emulsion for 24 h. 3 RHE are treated with base emulsion alone and 3 more remained untreated as a control batch (Ctrl). RHE are incubated under classical cell culture conditions (37° C., 5% CO₂).

Sample preparation and LC-MS/MS analysis: Protein is extracted using the Pierce Total Protein Extraction Kit (Thermo Fisher) according to manufacturer's recommendations. The protein is mixed with Laemmli buffer and heated to 37° C. for 30 min. Protein concentration is determined by the BCA method and standardized for all samples. The samples are separated by SDS-PAGE and digested with trypsin overnight. The peptides generated are acidified and separated. The mass spectra are queried using Proteome Discoverer (version 2.5). The resulting MS/MS data are queried against the Homo sapiens proteome UP000005640 (20371 reviewed entries). Results are filtered based on unique peptides >2, and global peptide scores. Analyses are performed using Proteome Discoverer 2.5 to check for overall sample quality.

Bioinformatics and statistical analysis: Proteomic analysis returned a mean total of 34961 high-quality peptides corresponding to a mean 2422 identifiable and quantifiable RHE proteins among conditions. A summary of proteins differentially expressed within conditions is provided in Annex 3. Proteins with a p-value 0.05 in at least one of the concentrations tested for a lipid are considered significant and used for pairwise comparisons. Relevant proteins are classified by their biological processes and associated pathways using the publicly available gene ontology (GO) database provided by the Gene Ontology Consortium and Reactome. Interactome analysis was performed using Cytoscape combined with STRING.

### Example 2: Characterisation of Anti-Aging Effects of Cer NS in Living Human Skin Explants

The aim of the study is to evaluate the effect of Cer NS on aging processes using living human skin explants. The activity is evaluated by immunostaining of laminin-5 and of collagen IV.

Collagen IV is the primary collagen found in the extracellular basement membranes separating a variety of epithelial and endothelial cells. It is a major component of the dermal-epidermal junction, where it is mostly found in the lamina densa. The laminins are also contained in the basal layer and participate in anchoring the surfaces of the cells to the basal lamina. Laminin-5 is a major hemidesmosomal component of the skin basement membrane Together, these two essential components of the DEJ ensure that the keratinocytes of the basal lamina are better anchored and contribute to maintain the suppleness of the epidermis.

### 2.1 Explant Preparation:

Human skin explants of an average diameter of 12 mm (±1 mm) were prepared on an abdoplasty coming from a 37-year-old Caucasian woman with a phototype II according to Fitzpatrick skin colour classification. The explants were kept in survival in BEM culture medium (BIO-EC's Explants Medium) at 37° C. in a humid, 5%—CO₂ atmosphere.

TABLE 1
Explant distribution:

| | | No of | |
|---|---|---|---|
| Batch | Conditions | explants | Sampling |
| T0 | Tissue control | 3 | Day 0 |
| T | Non-treated control | 6 | Day 4, Day 8 |
| E | Basic emulsion from Cosmacon GmbH | 6 | Day 4, Day 8 |
| | (composition in Table 2) | | |
| P | Basic emulsion from Cosmacon GmbH | 6 | Day 4, Day 8 |
| | with 0.1% CNS18 | | |

TABLE 2
Composition of basic emulsion from Cosmacon GmbH:

| Phase | Raw ingredient | INCI |
|---|---|---|
| Oily phase | Lanette 18 | Stearyl alcohol |
| | Emulgade PL 68/50 | Cetearyl Alcohol, Cetearyl |
| | | Glucoside |
| | Eutanol G | Octyldodecanol |
| Water phase | Water, demin | Aqua |
| | Glycerin 99.5% | Glycerin, Aqua |
| Thickening | Microcare Emollient PTG | Pentylene Glycol |
| phase | Keltrol CG -SFT V | Xanthan Gum |

### 2.2 Product Application:

The tested products E and P were applied topically at a rate of 2 L per 1 cm² explant (≈2 mg/cm²), and spread using a small spatula on day 0 (D0), D2, D4 and D6. The control explants T did not receive any treatment except the renewal of culture medium. The culture medium was half renewed (l ml per well) on D2, D4 and D6.

### 2.3 Sampling:

On D0, the 3 explants from the batch TO were collected and cut into two parts. Half was fixed in buffered formalin solution, one third was frozen at −80° C. On D4 and D8, 3 explants from the concerned batches were collected and treated in the same way as in D0.

### 2.4 Histological Processing and Cell Viability:

After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost® plus silanized glass slides. The microscopical observations were realised using a Leica DMLB, an Olympus BX43 or BX63 microscope. Pictures were digitized with a numeric DP72 or DP74 Olympus camera with CellSens storing software.

The cell viability of the epidermal and dermal structures was assessed by microscopical observation of formalin-fixed paraffin-embedded (FFPE) skin sections after Masson's trichrome staining, Goldner variant. The staining was assessed by microscopical observation.

### 2.4.1 Laminin-5 Immunostaining:

Laminin 5 immunostaining has been performed on frozen skin sections with a monoclonal anti-laminin 5 antibody (Santa Cruz, ref. sc-13587, clone P3E4), diluted at 1:100 in PBS-BSA 0.3%-Tween 20 (0.05%), for 1 hour at room temperature and revealed by AlexaFluor488 (Lifetechnologies, ref. A11001). The immunostaining was assessed by microscopical observation.

### 2.4.2 Collagen IV Immunostaining:

Collagen IV immunostaining was performed on frozen skin sections with a monoclonal anti-collagen IV antibody (Dako, ref M0785, clone CIV 22) diluted at 1:25 in PBS-BSA 0.3%-Tween 20 at 0.05% and incubated for 1 h at room temperature. The staining was revealed by AlexaFluor 488 (Lifetechnologies, ref. A11008). The immunostaining was assessed by microscopical observation.

### 2.6 Results:

### 2.6.1 Cell Viability:

On D4 and D8, cell viability is good or fairly good in the epidermis and in the dermis. Product E induces no modification on the cell viability. Product P induces on D8 a slight alteration in the epidermis but not in the dermis.

### 2.6.2 Image Analysis of Laminin-5 and Collagen IV:

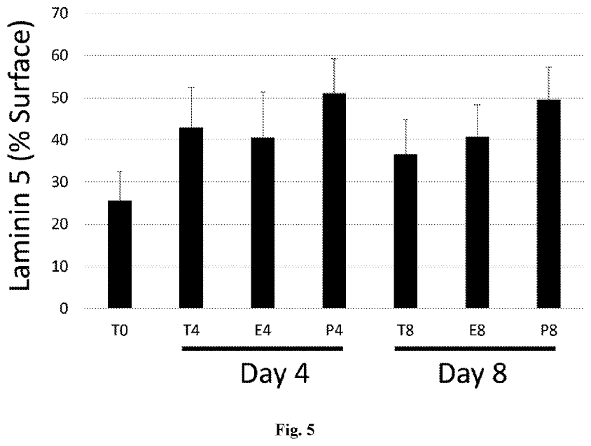

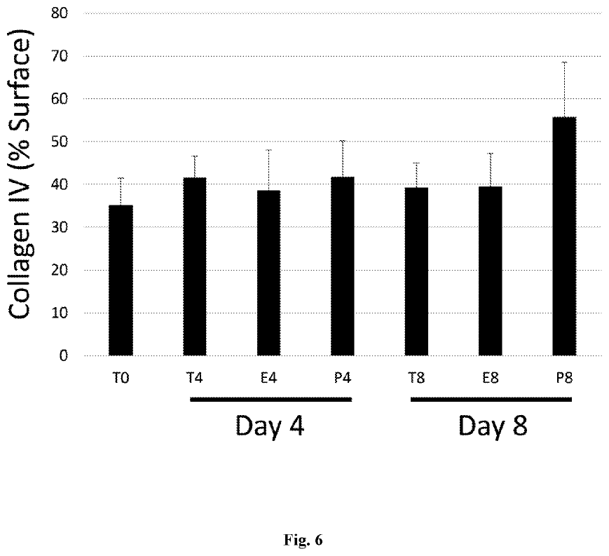

Treatment of living skin explants with CerNS results in significant increase in expression of laminin-5 (by 26%) already following 4 days of treatment, and of collagen IV (by 41% ) following 8 days of treatment. Results are also presented as graphs in FIGS. 5 and 6.

### Example 3: Characterisation of Effect of GlcCNS on IL-1a Concentration Using Skin Explants

The aim of the study is to evaluate the effect of GlcCNS on IL-1a levels using living human skin explants. The levels are evaluated by a biochemical quantification of IL-1a.

### 3.1 Explant Preparation:

Human skin explants of an average diameter of 11 mm (+1 mm) were prepared on an abdoplasty coming from a 57-year-old Caucasian woman with a II phototype according to Fitzpatrick skin colour classification. The explants were kept in survival in BEM culture medium (BIO-EC's Explants Medium) at 37° C. in a humid, 5%—CO₂ atmosphere.

TABLE 5
Explants distribution:

| | | Number of | |
|---|---|---|---|
| Batch | Conditions | explants | Sampling |
| T0 | Tissue control | 3 | Day 0 |
| T | Non-treated control | 4 | Day 6 |
| E | Basic emulsion from Cosmacon GmbH | 4 | Day 6 |
| P | Basic emulsion from Cosmacon GmbH | 4 | Day 6 |
| | with 0.05% GlcCNS18 | | |

### 3.2 Product Application:

The tested products E and P were applied topically at a rate of 2 μL per 1 cm² explant (2 mg/cm²), and spread using a small spatula on day 0 (D0), D1, D2 and D5. The control explants T did not receive any treatment except the renewal of culture medium. The culture medium was half renewed (1 ml per well) on D1, D2 and completely renewed (2 mL per well) on D5.

### 3.3 Sampling:

On D0, the 3 explants from the batch TO were collected and cut into two parts. One part was fixed in buffered formalin solution, and the other part was frozen at −80° C. for histological analysis. On D6, 4 explants from the concerned batches were collected and treated in the same way than in D0. The culture medium BEM of all batches was collected on D6 (2 mL per explant) and stored at −20° C. for dosages.

### 3.4 Histological Processing and Cell Viability:

After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The microscopical observations were realized using a Leica DMLB, an Olympus BX43 or BX63 microscope. Pictures were digitized with a numeric DP72 or DP74 Olympus camera with cellSens storing software (Olympus).

The cell viability of the epidermal and dermal structures was assessed by microscopical observation of formalin-fixed paraffin-embedded (FFPE) skin sections after Masson's trichrome staining, Goldner variant. The staining was assessed by microscopical observation.

3.5 IL-1α biochemical quantification:

IL-1α dosage was performed with human IL-1α ELISA kit (Cayman, ref 583301). According to provider's guidelines, the culture medium and the IL-1α standard were incubated with acetylcholinesterase (AChE) Fab′ conjugate binding IL-1α in wells which contain coated IL-1α antibody, overnight at 4° C. After well plate washing, the reaction was revealed with a solution containing AChE substrate. Absorbance at 412 nm was measured using the M200Pro Tecan microplate reader and Magellan7 software.

IL-1α assay was performed on culture BEM medium on day 6. The concentration of IL-1α was expressed in pg/ml.

### 3.6 Results:

### 3.6.1 Cell Viability:

On D0 and D6, cell viability is good in the epidermis and in the dermis. Products E and P induce no modification on the cell viability.

### 3.6.2 Dosage of IL-1a:

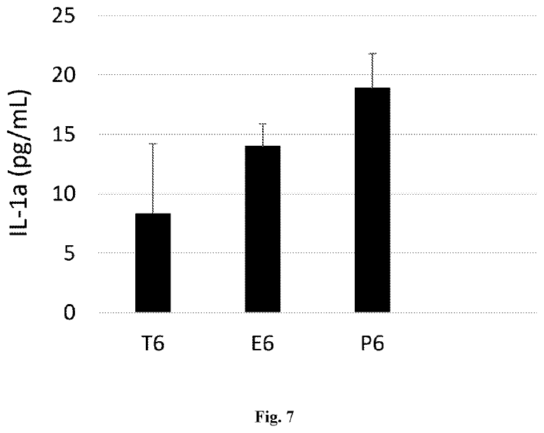

GlcCNS induces a significant increase on IL-1α release after 6 days. Results are also presented as graphs in FIG. 7.

The above described embodiments are combinable.

The following claims further set out particular embodiments of the disclosure.

1. Cosmetic use of a composition for reducing and/or ameliorating signs of skin aging in an individual, wherein the composition comprises a compound of formula I 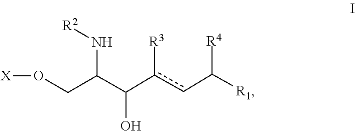
wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.

2. The cosmetic use according to claim 1, wherein-the composition is a topical or an oral composition.

3. The cosmetic use according to claim 1, wherein R¹ of the compound of formula I is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, and 30-(linoleyloxy)-triacontanoyl.

4. The cosmetic use according to claim 1, wherein the compound of formula I is present in the composition in an amount of about 0.0001% to about 10% wt/wt.

5. The cosmetic use according to claim 1, wherein the composition comprises:
(a) a compound of formula I wherein X is lactosyl and R² is a substituted or unsubstituted acyl having 16-34 carbon atoms;
(b) a compound of formula I wherein the carbon-carbon bond noted [symbol] is a single bond, X is hydrogen, R¹ is —C₁₂H₂₅, R² is an unsubstituted acyl having 16-34 carbon atoms, and R³ is a hydroxyl group;
(c) a compound of formula I wherein the carbon-carbon bond noted [symbol] is a single bond, X is hydrogen, R¹ is —C₁₂H₂₅, R² is an unsubstituted acyl having 16-34 carbon atoms, and R³ is hydrogen;
and/or
(d) a compound of formula I wherein the carbon-carbon bond noted [symbol] is a double bond, X is hydrogen, R¹ is —C₁₂H₂₅, and R² is an unsubstituted acyl having 16-34 carbon atoms.

6. The cosmetic use according to claim 1, comprising an increase of expression levels of one or more of the following: a structural protein of the skin,
a protein involved in cell adhesion, a protein involved in cellular communication and of Interleukin-1a (IL-1a) by keratinocytes.

7. The cosmetic use according to claim 1, wherein reducing and/or ameliorating signs of skin aging comprises one or more of the following: strengthening the skin structure, promoting cell adhesion, and promoting cell communication.

8. The cosmetic use according to claim 1, having one or more of the following effects on the skin of the individual:

| | |
|---|---|
| [[-]](a) | Improving skin firmness, |
| [[-]](b) | Increasing volume of the skin, |
| [[-]](c) | Improving skin homogeneity, |
| [[-]](d) | Improving skin complexion, |
| [[-]](e) | Improving skin radiance, and |
| [[-]](f) | Increasing skin hydration. |

9. The cosmetic use according to claim 1, wherein reducing and/or ameliorating signs of skin aging comprises reducing or ameliorating one or more of the following signs on the skin:

| | |
|---|---|
| [[-]](a) | wrinkles, |
| [[-]](b) | thinning, |
| [[-]](c) | decrease of elasticity, |
| [[-]](d) | sagging, |
| [[-]](e) | fine lines, |
| [[-]](f) | hyperkeratosis, |
| [[-]](g) | dry skin, |
| [[-]](h) | loss of skin volume, |
| [[-]](i) | stretch marks, and |
| [[-]](j) | sensitivity. |

10. Method for reducing and/or ameliorating signs of aging on skin of an individual, the method comprising administering to the individual a composition comprising a compound of formula I 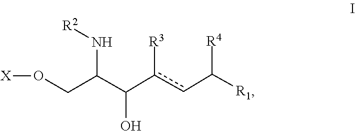
wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond,
X is hydrogen or a glycosyl moiety, wherein the glycosyl moiety is a lactosyl moiety or a glucosyl moiety.

11. The method according to claim 10, wherein the compound of formula I is an anti-aging compound.

12. The method according to claim 10, wherein the composition comprises one or more of the following: hyaluronic acid, melatonin, a peptide imitating a functional sequence of a structural protein, collagen, elastin, an anti-oxidant, a vitamin, a carotenoid, a polyphenol, coenzyme Q10, and azelaic acid.

13. The method according to claim 10, wherein the individual is a human individual.

14. The method according to claim 10, wherein the individual has one or more of the following:

| | |
|---|---|
| [[-]](a) | exposure to an environmental stressor, |
| [[-]](b) | heredity of premature skin aging, and |
| [[-]](c) | an unhealthy lifestyle. |

15. The method according to claim 14, wherein the environmental stressor comprises one or more of the following: UV light, chemical pollutants, polycyclic aromatic pollutants (PAH), particulate matter PM2.5, blue light, ozone, and cigarette smoke.

16. The method according to claim 14, wherein the unhealthy lifestyle comprises lack of sleep, smoking, alcohol abuse, unbalanced diet, or a combination thereof.

17. The method according to claim 14, wherein the exposure to the environmental stressor comprises a past, present, and/or future exposure.

18. The method according to claim 10, wherein the composition is administered to an individual for at least 2 days, at least 7 days, or at least 14 days.

19. (canceled)

20. (canceled)

21. The cosmetic use according to claim 1, wherein the compound of formula I is present in the composition in an amount from about 0.005% to about 5% wt/wt, or from about 0.05% to about 0.2% wt/wt.

22. The cosmetic use according to claim 6, wherein the protein comprises one or more of the following: a protein of the dermal-epidermal junction (DEJ), ECM1, Syndecan-1, Integrin alpha-3, Laminin-5, and Plakophilin-2.
